(19) United States
(12) Patent Application Publication
(10) Pub. No.: US 2024/0115770 A1
(43) Pub. Date: Apr. 11, 2024

(54) STIFFNESS TUNABLE BIOMIMETIC DECELLULARIZED EXTRACELLULAR MATRIX-BASED MATERIAL SYSTEMS

(71) Applicant: The Trustees of the University of Pennsylvania, Philadelphia, PA (US)

(72) Inventors: Su Chin Heo, Philadelphia, PA (US); Zizhao Li, Philadelphia, PA (US); Se-Hwan Lee, Philadelphia, PA (US)

(21) Appl. No.: 18/482,096

(22) Filed: Oct. 6, 2023

Related U.S. Application Data
(60) Provisional application No. 63/413,792, filed on Oct. 6, 2022.

(51) Int. Cl. A61L 27/52; A61L 27/20; A61L 27/38; C12M 1/12

(52) U.S. Cl. CPC A61L 27/52; A61L 27/20; A61L 27/38; C12M 25/14

(57) ABSTRACT

The present disclosure provides a tunable porous fibrous scaffold composition, comprising one or more modified glycosaminoglycans; and (ii) one or more extracellular matrix (ECM) proteins. The present disclosure also provides methods for preparing the tunable porous fibrous scaffold composition described herein.

### CROSS REFERENCE TO RELATED APPLICATIONS

This application claims priority to and the benefit of U.S. Provisional Patent Application No. 63/413,792 (filed Oct. 6, 2022), which is incorporated by reference herein in its entirety for any and all purposes.

### GOVERNMENT RIGHTS

This invention was made with government support under Contract No. AR07770 awarded by the National Institutes of Health. The government has certain rights in the invention.

### BACKGROUND

The human musculoskeletal system (MSK) is made up of bones, muscles, tendons, ligaments, cartilages, and other connective tissues that contribute significantly to the body's motion, stability, and protection of internal organs. Musculoskeletal disorders (MSD), defined as injuries or pain in the MSK, affect at least 112 million adult people in the United States, accounting for one third of workforce absence and costing 7.4 percent of GDP. Despite expansion of new tissue engineering technologies for MSD, the heterogeneity of MSK presents a major challenge in mimicking its complex biological and biomechanical environments. Thus, there is a need for stiffness-tunable biomimetic decellularized extracellular matrix-based material systems to recapitulate zonal dependent biomaterial and biomechanical properties to specifically enhance the heterogeneous dense connective MSK tissues (including tendons, ligaments, menisci, and articular cartilage).

### SUMMARY

In meeting the long-felt needs described above, the present disclosure provides a fibrous scaffold that includes (i) one or more modified glycosaminoglycans; and (ii) one or more extracellular matrix (ECM) proteins. In some embodiments, the fibrous scaffold includes a hydrogel.

Also provided is a method of preparing a fibrous scaffold, comprising: a) combining a decellularized ECM (dECM) solution and one or more modified glycosaminoglycans to give rise to a polymer solution; and b) generating a fibrous hydrogel scaffold from the polymer solution.

Further provided is a method for preparing a tissue biomimetic, comprising: a) seeding a plurality of cells into a fibrous scaffold as contemplated herein; and b) incubating the seeded fibrous scaffold for a period of time. In some embodiments, the plurality of cells comprises any one or more of tenocytes, osteoblasts, chondrocytes, or mesenchymal stem cells.

Also disclosed is an implantable article, comprising the fibrous scaffold as described herein.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1 depicts an exemplary model of a stiffness tunable tendon dECM and MeHA based bi-phasic scaffold for tendon-bone interface (e.g., rotator cuff enthesis) repair or regeneration as contemplated by the present disclosure.

FIG. 2A is a schematic showing the procedure of the fabrication of a biomimetic matrix-based bi-phasic scaffold system (BMS) for tissue repair including rotator cuff enthesis repair. FIG. 2B is a schematic showing a procedure of the fabrication of stiffness-tunable and alignment-tunable dECM/MeHA nanofibrous scaffolds.

FIG. 3A depicts H&E and PSR images taken before and after the decellularization of bovine Achilles tendons (arrows indicate the cell nuclei). FIG. 3B depicts representative SEM images of tendon dECM/MeHA nanofibrous scaffolds. FIG. 3C depicts the compressive moduli of scaffolds (n>30 points/group, mean±SEM). FIG. 3D depicts the tensile elastic moduli of scaffolds (n=4, mean±SEM, *: p<0.05, vs. 35% dE/M AL, ∘: p<0.05, vs 35% dE/M NAL, +: p<0.05, vs 100 dE/M AL, #: p<0.05, vs 100 dE/M NAL).

FIG. 4A depicts Picrocirius red (PSR) (top) and Alcian blue (AB) (bottom) images of MeHA alone or tendon dECM/MeHA fibrous scaffolds (bar=100 μm). FIG. 4B depicts scaffolds with DTAF (left) or PSR (right) staining after 7 days incubation in basal growth media.

FIG. 5A depicts representative F-actin staining images of bovine tenocytes spreading on nanofibrous scaffolds at day 3 and day 7 (bar=50 μm, arrows indicate the nanofiber direction). FIG. 5B depicts relative bovine tenocyte aspect ratio and circularity at day 3 under 5 different scaffold conditions (n=50 μm, mean±SEM, *: p<0.05, vs. 35% dE/M AL, ∘: p<0.05, vs 35% dE/M NAL, +: p<0.05, vs 100 dE/M AL, #: p<0.05, vs 100 dE/M NAL).

FIG. 6A depicts bovine tendon cell and FIG. 6B depicts bovine MSC proliferation on scaffolds determined by CCK8 assay (n=5/group, mean±SEM, *: p<0.05, vs. Day 1, #: p<0.05, vs. Day 3). FIG. 6C depicts gene expression relative to GAPDH in bovine tenocytes cultured on the scaffolds at day 7 (n=5/group, mean±SEM, *: p<0.01, **: p<0.001, ***: p<0.0001).

FIG. 7A depicts representative images of H&E (arrows: nuclei, scale bar: 200 μm), and quantifications of the number of nuclei in FDEM (ii), or in ADEM (iii). FIG. 7B Representative images of AB, and PSR (scale bar: 200 μm). FIG. 7C depicts images of fabricated age-dependent meniscus DEM hydrogels (i), Compressive moduli (ii). FIG. 7D depicts actin-stained images (i, scale bar: 100 μm), Relative cell area (ii), cell aspect ratio (iii). FIG. 7E depicts a graph of cell proliferation rates (*p<0.05, **p<0.01, ***p<0.001). FIGS. 7F and 7G are SEM images of FDEM (FIG. 7F) and ADEM (FIG. 7G) hydrogels and FIG. 7H is a graph showing the percentage of measured fiber diameters.

FIG. 8A shows relative gene expressions at day 5 (COL-II was not detected; *p<0.05, **p<0.01, ***p<0.001). FIG. 8B shows Proteomic analysis: (i) Heatmap of the differentially detected proteins in ADEM and FDEM, (ii) Relative enrichment ratios of remaining proteins in ADEM (compared to FDEM).

FIG. 9A shows proteomic analysis data (n=4 donors/group) from principal component analysis (PCA). FIG. 9B is an exemplary heatmap of the differentially detected proteins in ADEM and FDEM. FIG. 9C shows quantification of protein abundances in FDEM and ADEM (Top 20). FIG. 9D depicts GO analysis showing Number of genes (Top 10) related in each of the biological processes (a). FIG. 9E shows protein-protein networks of genes related with ECM structure, fibrochondrogenesis, cell adhesion, proliferation, and differentiation in FDEM, and FIG. 9F shows protein-protein networks of genes related with ECM structure, fibrochondrogenesis, cell adhesion, proliferation, and differentiation in ADEM.

FIG. 10A is a graph showing the compressive modulus of a stiffness tuned hydrogel system as contemplated by the present disclosure. FIG. 10B shows relative cell area and aspect ratio results from an in vitro bMSC cultured on a FADEM based MeHA hydrogel system. FIG. 10C shows YAP nuclear localization results and FIG. 10D shows gene expression results from an in vitro bMSC cultured on a FADEM based MeHA hydrogel system (*: p<0.05, **: p<0.01, ***: p<0.001, ns: not significant).

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Extracellular matrix (ECM) is a noncellular natural component that provides structural support and biochemical factors for living cells. Decellularized ECM (dECM) is composed of large molecules, such as collagen, elastin, fibronectin, laminin, and matricellular proteins. dECM materials have widespread applications for specific tissue or organ regeneration. Hyaluronic acid (HA) is one of the main components of the MSK soft tissues (e.g., cartilage, menisci, tendons) to connect the surrounding tissues. It has been shown that after electrospinning, methacrylate-modified HA (MeHA) materials can be firmly crosslinked with tunable mechanic properties to mimic tissue-specific heterogeneous fiber structures. Accordingly, embodiments of the disclosed technology are directed to dECM and MeHA fibrous combinations that possess abundant natural components with tunable stiffness, with the ability to be photo-crosslinked, and also demonstrate bioactivity for cellular attachment providing improvement for various tissue repair applications including rotator cuff enthesis repair and regeneration.

Porous Fibrous Scaffolds

The following provides exemplary, non-limiting embodiments of the disclosed technology. Any part of any embodiment can be combined with any part of any other embodiment.

The present disclosure provides, in some aspects, a biomimetic matrix-based multiphasic scaffold composition. The biomimetic matrix-based multiphasic scaffold composition can be a porous fibrous scaffold that is composed of a plurality of fibers having a plurality of orientations. The plurality of fibers can include one or more extracellular matrix proteins. The plurality of fibers can include one or more modified glycosaminoglycans. In some embodiments the ECM proteins include decellularized ECM proteins (dECM). For example, the dECM can include dECM derived from one or more sources including for example human, primate, bovine, equine, porcine, rattus, mus, camelid, canine, feline, ovine, leporine, and/or one or more combinations thereof. The dECM can be obtained from one or more tissue sources including tendon tissue. For example, the tissue can be sourced from one or more tendons including Achilles tendon, rotator cuff tendon, quadriceps tendon, flexor tendon, and the like. In some embodiments, the tissue can be sourced from one or more ligaments including for example lateral collateral ligament, medical collateral ligament, posterior cruciate ligament, and anterior cruciate ligament, and the like.

In some embodiments, the one or more ECM proteins include, for example, collagen, elastin, fibronectin, laminin, gelatin, matricellular proteins, and/or one or more combinations thereof. The collagen can include collagen I, collagen II, collagen III, collagen IV, collagen V, collagen VI, collagen X, collagen XI, collagen XII, collagen XIV, and one or more combinations thereof.

In some embodiments, the one or more modified glycosaminoglycans can include one or more of hyaluronic acid, chondroitin sulfate, heparin, heparan sulfate, keratan sulfate, dermatan sulfate, and/or one or more combinations thereof. The glycosaminoglycans can be modified by one or processes, e.g., methacrylation. In some embodiments, the modified glycosaminoglycan is methacrylated hyaluronic acid.

In some embodiments, the methacrylated glycosaminoglycan includes methacrylated hyaluronic acid. In some embodiments, the methacrylated glycosaminoglycan includes methacrylated chondroitin sulfate. In some embodiments, the methacrylated glycosaminoglycan includes methacrylated heparin. In some embodiments, the methacrylated glycosaminoglycan includes methacrylated heparin sulfate. In some embodiments, the methacrylated glycosaminoglycan includes methacrylated keratin sulfate. In some embodiments, the methacrylated glycosaminoglycan includes methacrylated dermatan sulfate.

In some embodiments, the methacrylated hyaluronic acid is methacrylated in the range of from about 0% to about 100%. In some embodiments, methacrylated hyaluronic acid is methacrylated in the range of from about 1% to about 50%, from about 5% to about 45%, from about 10% to about 40%, from about 15% to about 35%, from about 20% to about 30%, and any and all increments therebetween. In some embodiments, the methacrylated hyaluronic acid is methacrylated about 35%. In some embodiments, the methacrylated hyaluronic acid is methacrylated in the range of from about 50% to about 100%, from about 55% to about 95%, from about 60% to about 90%, from about 65% to about 85%, from about 70% to about 80%, and any and all increments therebetween. In some embodiments, the methacrylated hyaluronic acid is methacrylated about 100%. In some embodiments, the methacrylated hyaluronic acid is methacrylated in the range of from about 35% to about 100%.

In some embodiments, the porous fibrous scaffold has a first end and a second end wherein the methacrylated hyaluronic acid is methacrylated by about 35% at the first end and the methacrylated hyaluronic acid is methacrylated by about 100% at the second end. In some embodiments, the methacrylated hyaluronic acid is methacrylated in the range of from about 25% to about 45% at the first end. In some embodiments, the methacrylated hyaluronic acid is methacrylated in the range of from about 20% to about 50% at the first end. In some embodiments, the methacrylated hyaluronic acid is methacrylated in the range of from about 95% to about 100% at the second end. In some embodiments, the methacrylated hyaluronic acid is methacrylated in the range of from about 90% to about 100% at the second end. In some embodiments, the methacrylated hyaluronic acid is methacrylated in the range of from about 85% to about 100% at the second end. In some embodiments, the methacrylated hyaluronic acid is methacrylated in the range of from about 80% to about 100% at the second end. In some embodiments, the methacrylated hyaluronic acid is methacrylated in the range of from about 75% to about 100% at the second end.

In some embodiments, the amount of methacrylation directly relates to the rigidity of the scaffold. For example, the one or more regions of the porous fibrous scaffold that include modified glycosaminoglycans with a larger amount of methacrylation have a greater rigidity than the one or more regions of the porous fibrous scaffold that include modified glycosaminoglycans with a lesser amount of methacrylation. For example, regions of the porous fibrous scaffold that are methacrylated in the range of from about 20% to about 50% are less rigid that regions of the porous fibrous scaffold that are methacrylated in the range of from about 50% to about 100%. In some embodiments, the rigidity of the porous fibrous scaffold can be controlled or tuned by modifying the amount of methacrylation of the porous fibrous scaffold, including in one or more regions of the porous fibrous scaffold.

In some embodiments, the plurality of fibers of the porous fibrous scaffold have a plurality of orientations. In certain embodiments, the fibers have an aligned orientation. In certain embodiments, the fibers have a non-aligned orientation. In certain embodiments, the fibers have a combination of aligned and non-aligned orientations. For example, in some embodiments the fibers at the first end of the porous fibrous scaffold have an aligned orientation and the fibers at the second end have a non-aligned orientation. In some embodiments, the fibers at the first end have a more aligned orientation than the fibers at the second end. In some embodiment, the fibers at the first end have a more aligned or significantly aligned orientation and transition to a less aligned or non-aligned orientation or random orientation towards the second end. In some embodiments, the fibers at the second end have a non-aligned or significantly random orientation at the second end and transition to a more aligned or significantly aligned orientation towards the first end. In some embodiments, the porous fibrous scaffold includes a continuous range of from aligned to non-aligned orientations from the first end to the second end. In some embodiments, the porous fibrous scaffold includes at least two discrete regions having differently aligned orientations. For example, in some embodiments the porous fibrous scaffold includes a first discrete region having fibers with a significantly aligned orientation and a second discrete region having fibers with a significantly non-aligned orientation. In some embodiments, the porous fibrous scaffold includes two or more discrete regions with fibers having various degrees of alignment wherein when moving from one adjacent discrete region to the next, the fibers are increasingly aligned or decreasingly aligned when advancing from a first discrete region to one or more adjacent discrete regions to a final discrete region of the composition where the orientation of the fibers in the final discrete region are an opposite extreme from the orientation of the fibers in the first discrete region. In some embodiments, the amount of methacrylation is directly related to the amount of alignment of the fibers. For example, regions of the porous fibrous scaffold having a greater amount of methacrylation also have fibers that have a more aligned orientation. That is, a greater amount of methacrylation results in a greater amount of alignment of fibers to be produced when the polymer solution is crosslinked to form the porous fibrous scaffold. Similarly, a lesser amount of methacrylation results in a lesser amount of alignment of fibers to be produced when the polymer solution is crosslinked to form the porous fibrous scaffold.

In some embodiments, the one or more regions of the porous fibrous scaffold having fibers with an aligned orientation are more rigid than the one or more regions of the porous fibrous scaffold having fibers with a non-aligned orientation. For example, the one or more regions of the porous fibrous scaffold having fibers with an aligned orientation can have an elastic modulus ranging from about 30 MPa to about 80 MPa, from about 35 MPa to about 75 MPa, from about 40 MPa to about 70 MPa, from about 45 MPa to about 65 MPa, from about 50 MPa to about 60 MPa, and any and all increments therebetween. In some embodiments, the one or more regions of the porous fibrous scaffold having fibers with a non-aligned orientation are less rigid than the porous fibrous scaffold having fibers with an aligned orientation. For example, the one or more regions of the porous fibrous scaffold having fibers with a non-aligned orientation can have an elastic modulus ranging from about 1 MPa to about 30 MPa, from about 5 MPa to about 25 MPa, from about 10 MPa to about 20 MPa, and any and all increments therebetween.

In some embodiments, the porous fibrous scaffold has a cross-sectional dimension in the range of from about 0.5 nm to about 2 mm, from about 1 nm to about 1 mm, from about 10 nm to about 0.5 mm, and any and all increments therebetween.

Fibrous Sheets

The following provides exemplary, non-limiting embodiments of the disclosed technology. Any part of any embodiment can be combined with any part of any other embodiment.

The present disclosure provides a fibrous sheet scaffold composition. In certain embodiments, the fibrous sheet scaffold comprises a plurality of fibers having a plurality of orientations. Embodiments of the fibers can include one or more modified glycosaminoglycans. The fibrous sheet scaffold is also composed of one or more extracellular matrix (ECM) proteins. In some embodiments the ECM proteins include decellularized ECM proteins (dECM). For example, the dECM can include dECM derived from one or more sources including for example human, primate, bovine, equine, porcine, rattus, mus, camelid, canine, feline, ovine, leporine, and/or one or more combinations thereof. The dECM can be obtained from one or more tissue sources including tendon tissue, ligament tissue, one or more cartilaginous tissues, and the like. For example, the one or more tendon tissues can include the Achilles tendon, rotator cuff tendon, quadriceps tendon, flexor tendon, and the like. The one or more ligament tissues can include, for example, lateral collateral ligament, medical collateral ligament, posterior cruciate ligament, and anterior cruciate ligament, and the like. The one or more cartilaginous tissues can include, for example the meniscus, vertebral discs, and the like.

In some embodiments, the fibrous sheet scaffold includes one or more ECM proteins including, for example, collagen, elastin, fibronectin, laminin, gelatin, matricellular proteins, and/or one or more combinations thereof. In some embodiments, the collagen includes collagen I, collagen II, collagen III, collagen IV, collagen V, collagen VI, collagen X, collagen XI, collagen XII, collagen XIV, and one or more combinations thereof.

In some embodiments, the fibrous sheet scaffold includes one or more modified glycosaminoglycans. The one or more modified glycosaminoglycans can include one or more of hyaluronic acid, chondroitin sulfate, heparin, heparan sulfate, keratan sulfate, dermatan sulfate, and/or one or more combinations thereof. The glycosaminoglycans can be modified by one or processes, e.g., methacrylation. In some embodiments, the modified glycosaminoglycan is methacrylated hyaluronic acid.

In some embodiments, the methacrylated glycosaminoglycan includes methacrylated hyaluronic acid. In some embodiments, the methacrylated glycosaminoglycan includes methacrylated chondroitin sulfate. In some embodiments, the methacrylated glycosaminoglycan includes methacrylated heparin. In some embodiments, the methacrylated glycosaminoglycan includes methacrylated heparin sulfate. In some embodiments, the methacrylated glycosaminoglycan includes methacrylated keratin sulfate. In some embodiments, the methacrylated glycosaminoglycan includes methacrylated dermatan sulfate.

In some embodiments, the fibrous sheet scaffold includes methacrylated hyaluronic acid that is methacrylated in the range of from about 0% to about 100%. In some embodiments, methacrylated hyaluronic acid is methacrylated in the range of from about 1% to about 50%, from about 5% to about 45%, from about 10% to about 40%, from about 15% to about 35%, from about 20% to about 30%, and any and all increments therebetween. In some embodiments, the methacrylated hyaluronic acid is methacrylated about 35%. In some embodiments, the methacrylated hyaluronic acid is methacrylated in the range of from about 50% to about 100%, from about 55% to about 95%, from about 60% to about 90%, from about 65% to about 85%, from about 70% to about 80%, and any and all increments therebetween. In some embodiments, the methacrylated hyaluronic acid is methacrylated about 100%. In some embodiments, the methacrylated hyaluronic acid is methacrylated in the range of from about 35% to about 100%. The fibrous sheet scaffold can include a single percentage of methacrylation. The fibrous sheet scaffold can include one or more percentages of methacrylation. The fibrous sheet scaffold can include a gradient of methacrylation ranging from about 0% to about 100%. The gradient can include increments of methacrylation of about 1%, about 2% about 3%, about 4% about 5% and the like. The fibrous sheet scaffold can include a gradient of methacrylation ranging from about 35% to about 100%, from about 10% to about 100%, from about 35% to about 75%, from about 10% to about 90%, and the like.

In some embodiments, the plurality of fibers of the fibrous sheet scaffold have a plurality of orientations. In certain embodiments, the fibers have an aligned orientation. In certain embodiments, the fibers have a non-aligned orientation. In certain embodiments, the fibers have a combination of aligned and non-aligned orientations. For example, in some embodiments the fibers at the first end of the porous fibrous scaffold have an aligned orientation and the fibers at the second end have a non-aligned orientation. In some embodiments, the fibers at the first end have a more aligned orientation than the fibers at the second end. In some embodiment, the fibers at the first end have a more aligned or significantly aligned orientation and transition to a less aligned or non-aligned orientation or random orientation towards the second end. In some embodiments, the fibers at the second end have a non-aligned or significantly random orientation at the second end and transition to a more aligned or significantly aligned orientation towards the first end. In some embodiments, the porous fibrous scaffold includes a continuous range of from aligned to non-aligned orientations from the first end to the second end. In some embodiments, the porous fibrous scaffold includes at least two discrete regions having differently aligned orientations. For example, in some embodiments the porous fibrous scaffold includes a first discrete region having fibers with a significantly aligned orientation and a second discrete region having fibers with a significantly non-aligned orientation. In some embodiments, the porous fibrous scaffold includes two or more discrete regions with fibers having various degrees of alignment wherein when moving from one adjacent discrete region to the next, the fibers are increasingly aligned or decreasingly aligned when advancing from a first discrete region to one or more adjacent discrete regions to a final discrete region of the composition where the orientation of the fibers in the final discrete region are an opposite extreme from the orientation of the fibers in the first discrete region.

In some embodiments, the one or more regions of the porous fibrous scaffold having fibers with an aligned orientation are more rigid than the one or more regions of the porous fibrous scaffold having fibers with a non-aligned orientation. For example, the one or more regions of the porous fibrous scaffold having fibers with an aligned orientation can have an elastic modulus ranging from about 30 MPa to about 80 MPa, from about 35 MPa to about 75 MPa, from about 40 MPa to about 70 MPa, from about 45 MPa to about 65 MPa, from about 50 MPa to about 60 MPa, and any and all increments therebetween. In some embodiments, the one or more regions of the porous fibrous scaffold having fibers with a non-aligned orientation are less rigid than the porous fibrous scaffold having fibers with an aligned orientation. For example, the one or more regions of the porous fibrous scaffold having fibers with a non-aligned orientation can have an elastic modulus ranging from about 1 MPa to about 30 MPa, from about 5 MPa to about 25 MPa, from about 10 MPa to about 20 MPa, and any and all increments therebetween.

Implantable Article

In certain aspects, the present disclosure relates to an implantable article comprising the porous fibrous scaffold as contemplated herein. In some embodiments, the implantable article is configured as an anchor, for example a tissue anchor for securing one or more first tissues to one or more second tissues. In some embodiments the one or more first tissues comprise one or more of a tendon, a ligament, cartilage, and the like. For example, the one or more first tissues can include one or more of an Achilles tendon, rotator cuff tendon, quadriceps tendon, flexor tendon, a lateral collateral ligament, medical collateral ligament, posterior cruciate ligament, anterior cruciate ligament, meniscus, and the like. In some embodiments the one or more second tissues can include one or more entheses including fibrous entheses and fibrocartilaginous enthesis. For example, the one or more second tissues can include one or more enthesis including one or more of a rotator cuff enthesis, Achilles tendon entheses, deltoid attachment to the humerus, Adductor magnus attachment to the linea aspera of the femur, pronator teres, and the like, as understood in the art.

In some embodiments, the porous fibrous scaffold as contemplated herein in combined with one or more anchoring devices as understood in the art. For example, in some embodiments the porous fibrous scaffold is combined with one or more of a bone anchor, a bone screw, a suture anchor, and the like.

In some embodiments, the implantable article is positioned so that the porous fibrous scaffold is oriented so that the end with the aligned fibers is in contact with or significantly adjacent to tendon tissue and/or ligament tissue. In some embodiments, the implantable article is positioned so that the porous fibrous scaffold is oriented so that the end with the non-aligned fibers is in contact with or significantly adjacent to bone tissue or one or more entheses.

In some embodiments, the implantable article is configured as a tissue biomimetic for tissue replacement or tissue repair. In some embodiments, the implantable article includes a porous fibrous scaffold constructed as a replacement for one or more fibrocartilaginous tissues, including for example a meniscus, a verbal disc, and the like. In such embodiments, the porous fibrous scaffold is prepared as a scaffold or fibrous sheet having one or more regions with an elastic modulus suitable to provide the mechanical properties needed for the designated tissue. For example, in some embodiments, the implantable article is configured as a meniscus mimetic with one or more regions with an elastic modulus similar to that of different regions of a native meniscus including the inner avascular (white-white) zone, intermediate transitional (red-white) zone, and outer vascularized (red-red) zone.

In some embodiments, the implantable article is an implanted porous fibrous scaffold having prescribed mechanical properties as described herein. In some embodiments, the implantable article is an implanted porous fibrous scaffold that is also seeded with one or more populations of cells. The cells can include any one or more of tenocytes, meniscus cells, chondrocytes, osteoblasts, mesenchymal stem cells, fibrochondrocytes, and one or more combinations thereof. The cells can be seeded at a density of up to about 30,000 cells/cm3, from about 30,000 cells/cm3 to about 50,000 cells/cm3, from about 50,000 cells/cm3 to about 100,000 cells/cm3, from about 100,000 cells/cm3 to about 500,000 cells/cm3, from about 500,000 cells/cm3 to about 1,000,000 cells/cm3, from about 1,000,000 cells/cm3 to about 2,000,000 cells/cm3, from about 2,000,000 cells/cm3 to about 5,000,000 cells/cm3, and any and all increments therebetween.

The population of cells can be seeded onto the porous fibrous scaffold for a period of time before implanting the implantable article. For example, the seeded scaffold composition including the porous fibrous scaffold, and the plurality of seeded cells can be incubated for a period of time including up to about 1 day, from about 1 day to about 3 days, from about 3 days to about 5 days, from about 5 days to about 7 days, from about 7 days to about 10 days, from about 10 days to about 12 days, from about 12 days to about 14 days, from about 14 days to about 21 days, from about 21 days to about 28 days, and any and all increments therebetween

Preparation of a Porous Fibrous Scaffold Composition

In certain aspects, the present disclosure relates to methods for preparing a porous fibrous scaffold, as contemplated herein.

Embodiments of the methods as contemplated herein include decellularizing an ECM-containing tissue thereby obtaining a decellularized ECM (dECM) solution. The ECM-containing tissue can include one or more of tendon tissue and/or ligament tissue. For example, the tissue can be sourced from one or more tendons including Achilles tendon, rotator cuff tendon, quadriceps tendon, flexor tendon, and the like. In some embodiments, the tissue can be sourced from one or more ligaments including for example lateral collateral ligament, medical collateral ligament, posterior cruciate ligament, and anterior cruciate ligament, and the like. The ECM-containing tissue can be decellularized using one or more techniques as understood in the art. For example, the tissue can be decellularized using one or more solutions such as 1% SDS in PBS for a period of up to about 3 days.

In some embodiments, the dECM includes one or more ECM proteins including for example, collagen, elastin, fibronectin, laminin, gelatin, matricellular protein, and/or one or more combinations thereof. In some embodiments, the collagen includes collagen I, collagen II, collagen III, collagen IV, collagen V, collagen VI, collagen X, collagen XI, collagen XII, collagen XIV, and one or more combinations thereof.

Embodiments of the methods include combining the dECM solution and one or more modified glycosaminoglycans in order to give rise to a polymer solution. In some embodiments, the one or more modified glycosaminoglycans includes one or more of hyaluronic acid, chondroitin sulfate, heparin, heparan sulfate, keratan sulfate, dermatan sulfate, and/or one or more combinations thereof. The glycosaminoglycans can be modified by one or processes including methacrylation. In some embodiments, the modified glycosaminoglycan is methacrylated hyaluronic acid.

In some embodiments, the methacrylated glycosaminoglycan includes methacrylated hyaluronic acid. In some embodiments, the methacrylated glycosaminoglycan includes methacrylated chondroitin sulfate. In some embodiments, the methacrylated glycosaminoglycan includes methacrylated heparin. In some embodiments, the methacrylated glycosaminoglycan includes methacrylated heparin sulfate. In some embodiments, the methacrylated glycosaminoglycan includes methacrylated keratin sulfate. In some embodiments, the methacrylated glycosaminoglycan includes methacrylated dermatan sulfate.

Embodiments of the methods include generating a porous fibrous scaffold from the polymer solution as contemplated herein. The polymer solution includes a dECM solution and one or more modified glycosaminoglycans, for example methacrylated hyaluronic acid. The generated porous fibrous scaffold can include a hydrogel scaffold. The porous fibrous scaffold is formed by crosslinking the polymer solution using one or more techniques including elevating temperature, photocrosslinking, and/or a combination thereof. For example, in some embodiments, the porous fibrous scaffold is formed by elevating the temperature of the polymer solution. In some embodiments, the porous fibrous scaffold if formed by photocrosslinking the polymer solution using light in the UV range of wavelengths on the electromagnetic radiation (EMR) spectrum. For example, the porous fibrous scaffold can be formed by exposing polymer solution to light having a wavelength in the range of from about 100 nm to about 400 nm, including for example 365 nm. The porous fibrous scaffold can be crosslinked by exposing the polymer solution to UV light for one or more durations of time including for example, up to 1 minute, from about 1 minute to about 5 minutes, from about 5 minutes to about 10 minutes, from about 10 minutes to about 15 minutes, from about 15 minutes to about 20 minutes, from about 20 minutes to about 25 minutes, from about 25 minutes to about 30 minutes, from about 30 minutes to about 35 minutes, from about 35 minutes to about 40 minutes, from about 40 minutes to about 45 minutes, from about 45 minutes to about 50 minutes, from about 50 minutes to about 55 minutes, from about 55 minutes to about 60 minutes, including any and all increments therebetween. In some embodiments, the hydrogel scaffold is formed by first crosslinking the polymer solution by increasing the temperature and then also exposing the polymer solution to UV light.

The porous fibrous scaffold can be generated using one or more techniques as understood in the art. For example, the porous fibrous scaffold can be generated by electrospinning the polymer solution. The electrospinning parameters can by adjusted in order to generate electrospun fibers having a diameter including for example a fiber diameter of up to about 0.05 μm, from about 0.05 μm to about 0.1 μm, from about 0.1 μm to about 0.2 μm, from about 0.2 μm to about 0.3 μm, from about 0.3 μm to about 0.4 μm, from about 0.4 μm to about 0.5 μm, from about 0.5 μm to about 0.6 μm, from about 0.6 μm to about 0.7 μm, from about 0.7 μm to about 0.8 μm, from about 0.8 μm to about 0.9 μm, from about 0.9 μm to about 1 μm, from about 1 μm to about 2 μm, and any and all increments therebetween.

In some embodiments, the porous fibrous scaffold can be generated by bioprinting the polymer solution. For example, the porous fibrous scaffold can be bioprinted in a pattern suitable to possess biomechanical properties desirable for one or more tissue applications including for example, a rotator cuff biomimetic, a meniscus biomimetic, and or a mimetic or repair for one or more other ligaments, tendons, cartilaginous tissues, and the like. The porous fibrous scaffold can be bioprinted by first loading polymer solution into a syringe equipped with a needle, or other appropriate dispensing device for printing as understood in the art. While maintaining the printer head of the bioprinting device and the polymer solution at temperature of about 4° C., the polymer solution can be printed onto a bed heated to a temperature of about 37° C. by extruding the polymer solution with pneumatic pressure. The bioprinted polymer can undergo a first temperature-dependent cross-linking step on the pre-heated printing bed. That is, the printed polymer solution undergoes an initial crosslinking step as the temperature of the solution is elevated from about 4° C. to about 37° C. as it comes into contact with the pre-heated printing bed. In some embodiments, the first, temperature-dependent crosslinking of the polymer solution occurs at a temperature less than 37° C. The bioprinted polymer can undergo a secondary cross-linking step by exposing the bioprinted polymer solution to UV light with a wavelength of about 365 nm.

In some embodiments, the resulting bioprinted scaffold can be seeded with cells once the polymer solution has formed a crosslinked scaffold. In some embodiments, the bioprinted polymer solution can include a solution of a population of cells that are bioprinted with the polymer solution. The one or more cells populations can be printed with or seeded on the bioprinted scaffold at one or more concentrations including for example, up to about 30,000 cells/cm³, from about 30,000 cells/cm³ to about 50,000 cells/cm³, from about 50,000 cells/cm³ to about 100,000 cells/cm³, from about 100,000 cells/cm³ to about 500,000 cells/cm³, from about 500,000 cells/cm³ to about 1,000,000 cells/cm³, from about 1,000,000 cells/cm³ to about 2,000,000 cells/cm³, from about 2,000,000 cells/cm³ to about 5,000,000 cells/cm³, and any and all increments therebetween.

In some embodiments, the porous fibrous scaffold can be generated by crosslinking an injectable hydrogel. The injectable hydrogel can include a polymer solution as contemplated herein. The injectable polymer solution can include cells. The injectable polymer solution can be cell-free. The injectable polymer solution can be maintained in solution at a temperature of about 4° C. prior to injection. The injectable polymer solution can be injected into a defect in one or more tissues such as a meniscus. The defect can include for example a tear. The injectable polymer solution can be injected into, for example, a tear or other defect in the torn or injured tissue, for example, meniscus, wherein the tissue is at a temperature greater than 4° C., including for example 37° C. The injectable polymer solution can undergo a first crosslinking step due to an increase in temperature of the polymer solution from 4° C. to a temperature of up to and including 37° C. The injected polymer solution can undergo a second crosslinking step wherein the injected polymer solution is exposed to UV light having a wavelength of about 365 nm. The second crosslinking step can including exposing the injected polymer solution to UV light for a duration of time including up to about including about 5 minutes, from about 5 minutes to about 10 minutes, from about 10 minutes to about 20 minutes, from about 20 minutes to about 30 minutes, from about 30 minutes to about 40 minutes, from about 40 minutes to about 50 minutes, from about 50 minutes to about 60 minutes, including any and all increments therebetween.

Tissue Biomimetic

In certain aspects, the present disclosure provides methods for preparing a tissue biomimetic.

Embodiments of the methods include obtaining porous fibrous scaffold as contemplated herein. For example, embodiments of the methods include obtaining a porous fibrous scaffold constructed of methacrylated hyaluronic acid and decellularized ECM including, for example collagen dECM. In some embodiments, the obtained porous fibrous scaffold is formed by electrospinning. In some embodiments, the porous fibrous scaffold is formed by bioprinting. In some embodiments, the porous fibrous scaffold is formed by crosslinking an injectable hydrogel.

Embodiments of the present methods include seeding a plurality of cells into the porous fibrous scaffold. The cells can include any one or more of tenocytes, meniscus cells, chondrocytes, osteoblasts, mesenchymal stem cells, fibrochondrocytes, and one or more combinations thereof. The cells can be seeded at a density of up to about 30,000 cells/cm³, from about 30,000 cells/cm³ to about 50,000 cells/cm³, from about 50,000 cells/cm³ to about 100,000 cells/cm³, from about 100,000 cells/cm³ to about 500,000 cells/cm³, from about 500,000 cells/cm³ to about 1,000,000 cells/cm³, from about 1,000,000 cells/cm³ to about 2,000,000 cells/cm³, from about 2,000,000 cells/cm³ to about 5,000,000 cells/cm³, and any and all increments therebetween.

Embodiments of the present methods include incubating the seeded porous fibrous scaffold for a period of time. For example, the seeded scaffold composition including the porous fibrous scaffold, and the plurality of seeded cells can be incubated for a period of time including up to about 1 day, from about 1 day to about 3 days, from about 3 days to about 5 days, from about 5 days to about 7 days, from about 7 days to about 10 days, from about 10 days to about 12 days, from about 12 days to about 14 days, from about 14 days to about 21 days, from about 21 days to about 28 days, and any and all increments therebetween.

The following Examples are provided to illustrate some of the concepts described within this disclosure. While the Examples are considered to provide embodiments, it should not be considered to limit the more general embodiments described herein.

Example 1—Rotator Cuff Tendon-Bone Enthesis Regeneration

Introduction

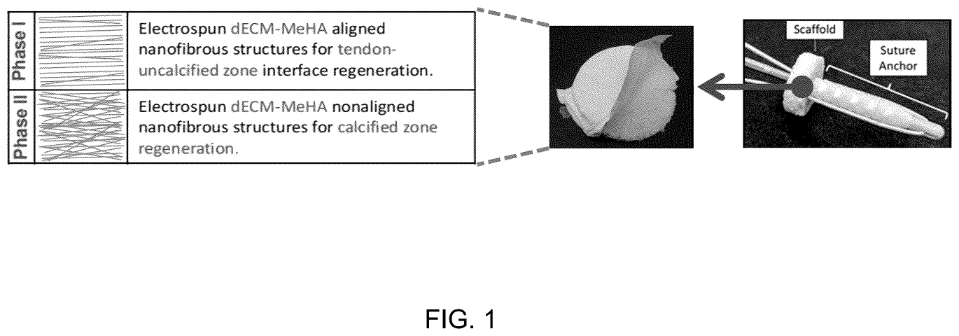

The rotator cuff plays an important role in shoulder movements and keeping shoulder joint stability. One feature of the rotator cuff tendon-bone interface (enthesis) is the heterogeneous structure with distinct cell phenotypes and ECM composition, where there are uncalcified (connected to tendon, rich in aligned Collagen-I and III, GAG, and fibrochondrocytes) and calcified fibrocartilage (connected to the bone, filled with non-aligned Collagen-I, -II and -X, GAG, hydroxyapatite, and hypertrophic fibrochondrocytes) zones in the enthesis (Apostolakos, et al. Muscles Ligaments Tendons Journal 2014). Tears often occur at the enthesis, resulting in pain, weakness, irritation, and reduced range of motion. Each year in the United States, the population size of rotator cuff tendon injuries is considerably large, with more than 300,000 cases that are under surgical treatments (Carr et al. Health Technology Assessment 2015). Nevertheless, the surgically treated tendon-bone interface often turns to re-tear owing to the formation of scar tissues that prevent the establishment of enthesis combination. Therefore, multiple new approaches are investigated and introduced into the rotator cuff enthesis repair field, including lab-designed or commercially available tissue-engineered scaffolds with the potential to improve the enthesis integration. However, the limitations of current tissue engineered devices are due to the scaffold materials without specific bioactive and biophysical cues and failing to establish the scaffolds with the native structural, biochemical, and mechanical characteristics of the native rotator cuff tendon enthesis. To overcome the drawbacks of current materials and to recapitulate zonal dependent biomaterial/biomechanical properties of the rotator cuff enthesis, a biomimetic matrix-based stiffness-tunable multiphasic scaffold system (BMS) was developed. The porous fibrous scaffold consists of the phase I [aligned bovine Achilles-tendon decellularized-extracellular matrix (dECM) scaffolds that are electrospun with 100% modified (‘stiff’) methacrylate hyaluronic acid (MeHA) for tendon-uncalcified zone regeneration], and the phase II [nonaligned dECM scaffolds that are electrospun with 35% modified (‘soft’) MeHA for tendon-calcified zone regeneration] (FIG. 1).

Methods

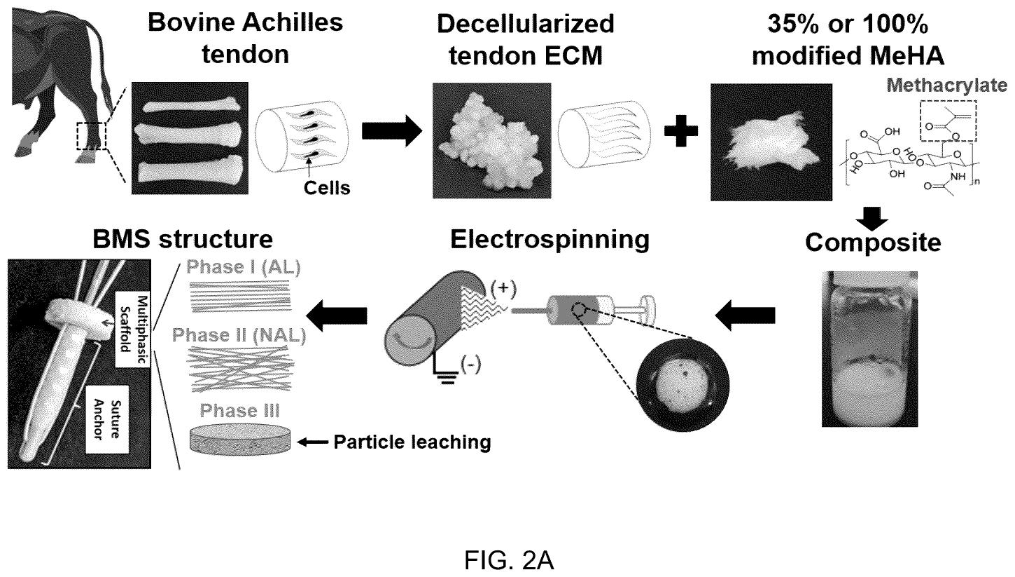

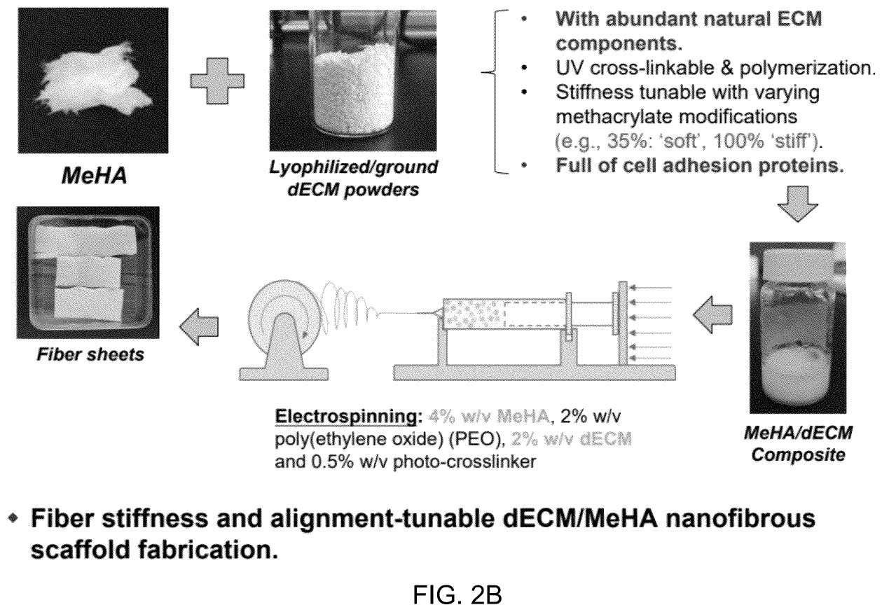

Juvenile bovine Achilles tendon was decellularized in SDS and EDTA-based solutions. Hematoxylin and eosin (H&E), and Picrocirius red (PSR) staining were utilized to confirm the decellularization accomplishment and tendon ECM preservation. dECM acidic solutions were prepared by dissolving dECM powder in acetic acid, ethyl acetate, and DI water solution at a volume rate of 3:2:1. To synthesize 35% (soft) or 100% (stiff) modified MeHA, 2.6 mL or 7.4 mL of methacrylic anhydride (Sigma) was added into 100 mL of a 1% (w/v) sodium hyaluronate solution. Stiff aligned (Phase I: uncalcified zone) or soft non-aligned (Phase II: calcified zone) nanofibrous scaffolds were fabricated via electrospinning with loaded 4% w/v stiff or soft MeHA, 2% w/v PEO (Acros Organics), 2% w/v dECM, and 0.5% w/v Irgacure 2959 (FIG. 2A, 2B). The mixed solution was loaded into a 20 mL plastic syringe with an 18-gauge needle and injected using a syringe pump at a flow rate of 1 mL/h. Both sides of scaffolds were exposed under 1.7 mW/cm² UV lights for 30 minutes to crosslink. The surface morphology of stiffness-tunable scaffolds was examined by the scanning electron microscopy (SEM) (FEI Quanta). The compressive moduli of fibers were evaluated by atomic force microscopy (AFM) and the uniaxial tensile tests were performed on electrospun dECM/MeHA scaffold strips (5×30 mm²) using an Instron. For further crosslinking, scaffolds were immersed in 0.4 M genipin (GP) ethanol solution at 37° C. for 48 hours. PSR, Alcian blue (AB), and 5-(4,6-dichlorotriazinyl) aminofluorescein (DTAF) staining were performed to validate the success of dECM preservation on scaffolds. Bovine tendon cells or bovine mesenchymal stem cells (MSCs) (Passage 1) were seeded on aligned (AL) or non-aligned (NAL), or stiff or soft dECM MeHA scaffolds, respectively and cultured in basal cell growth media (without any soluble growth factors) up. Cell attachment and proliferation rates were evaluated by Filamentous actin (F-actin) staining and Cell Counting Kit 8 (CCK8) assay, respectively. The cell aspect ratio and circularity of the cells cultured on scaffolds were calculated by ImageJ. The bovine tendon cell gene expression level of Collagen-IA2 and -III (fibrous marker), Tenomodulin and Tenascin C (tenogenic markers), Collagen-II and Aggrecan (chondrogenic markers), and GAPDH (a housekeeping gene) were measured at day 7 using real-time PCR technique.

Results

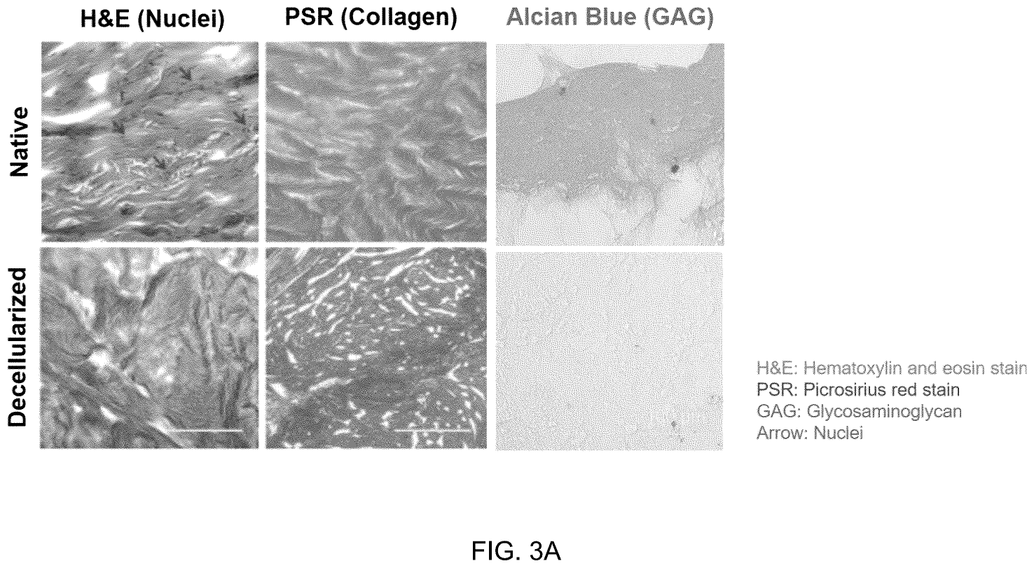

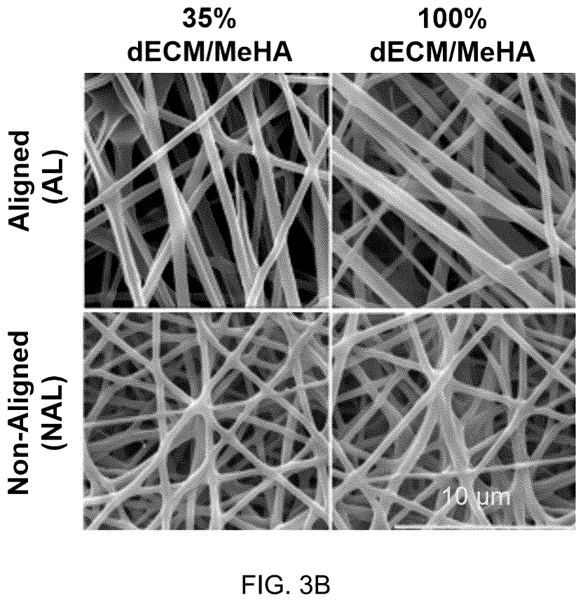

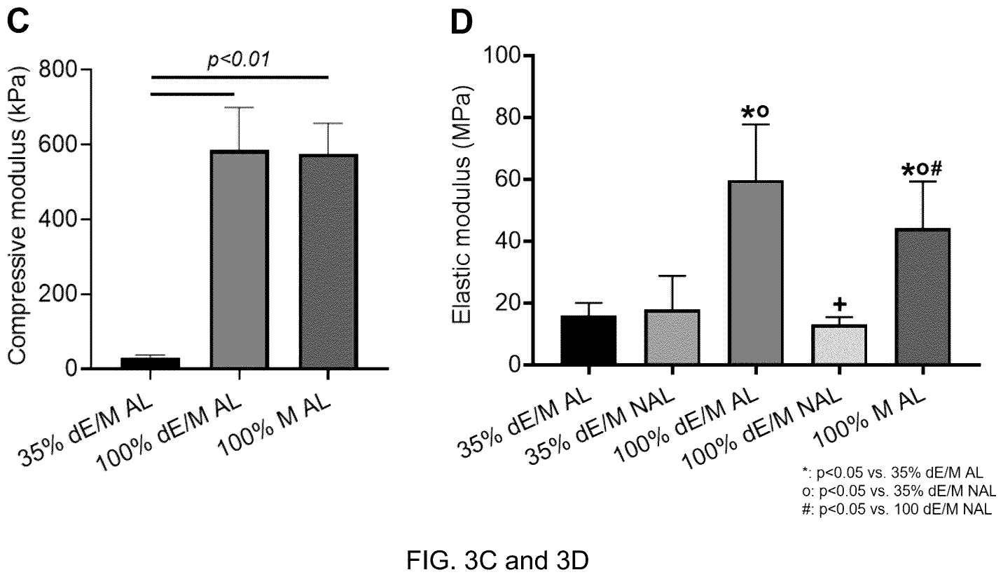

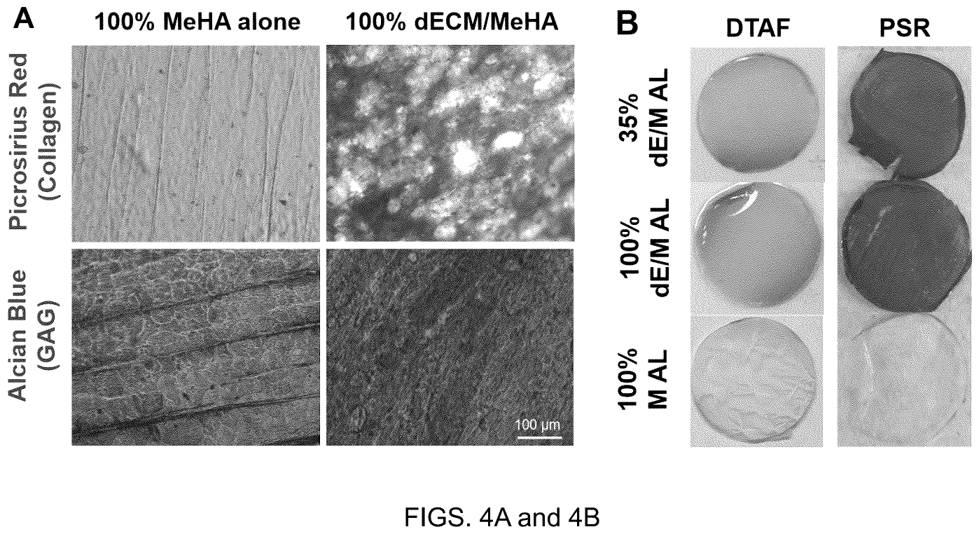

The loss of nuclei from decellularized Achilles tendon tissues was observed by H&E staining, and Picrocirius red confirmed that the collagen was preserved after the decellularization process (FIG. 3A). SEM images presented that aligned (AL) or non-aligned (NAL) electrospun 35% (soft)/100% (stiff) dECM/MeHA scaffolds were fabricated for the Phase I-II (FIG. 3B). The compressive moduli of aligned dECM/35% MeHA (35% dE/M AL), dECM/100% MeHA (100% dE/M AL), and 100% MeHA alone (100% M AL) fibers were determined by AFM as ˜29 kPa, ˜585 kPa, and ˜575 kPa respectively (FIG. 3C). FIG. 3D showed that 100% dE/M AL scaffolds have the highest elastic modulus indicating that higher MeHA modification rate increases fiber stiffness Alcian blue (AB) and Picrocirius red (PSR) staining revealed that aligned dECM/MeHA scaffolds had a certain amount of glycosaminoglycan and good collagen distribution when compared to the MeHA alone group (FIG. 4A). UV light and Genipin crosslinked MeHA/dECM scaffolds were stable after 7 days incubation in basal cell growth media, determined by DTAF and PSR staining (FIG. 4B).

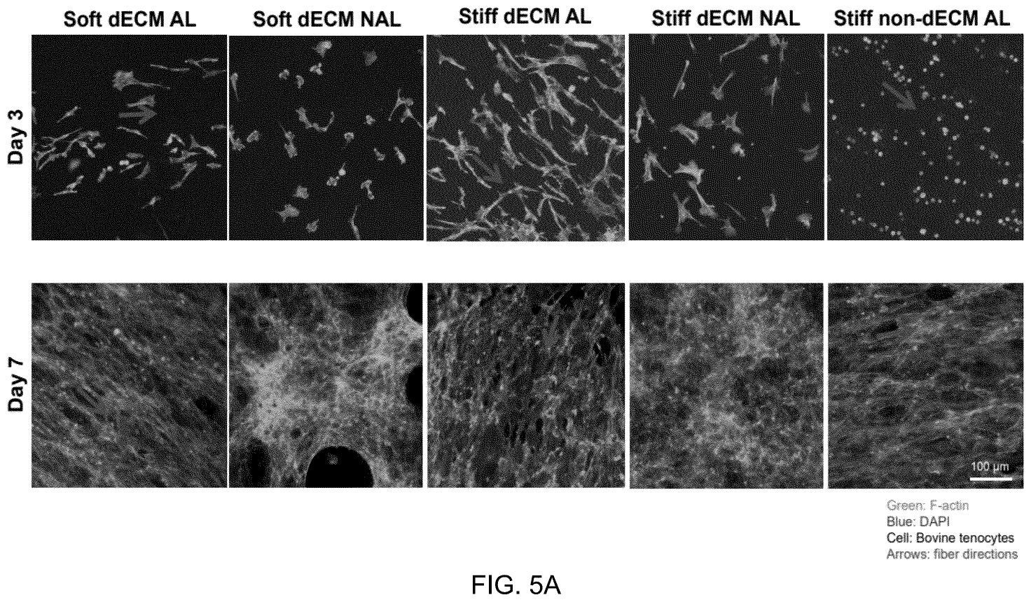

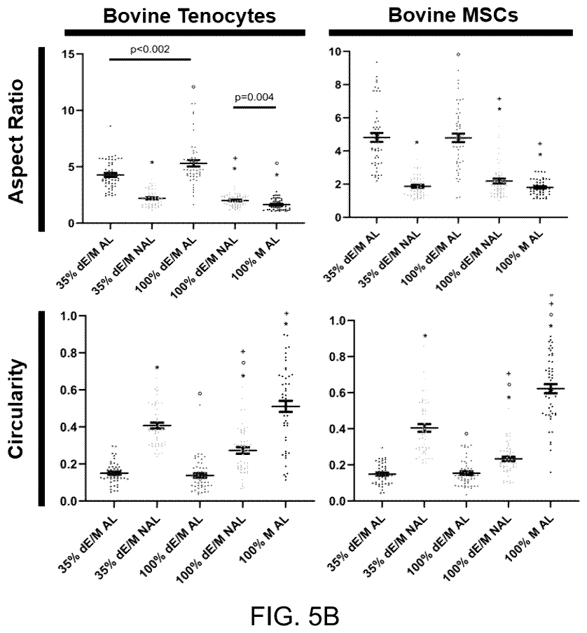

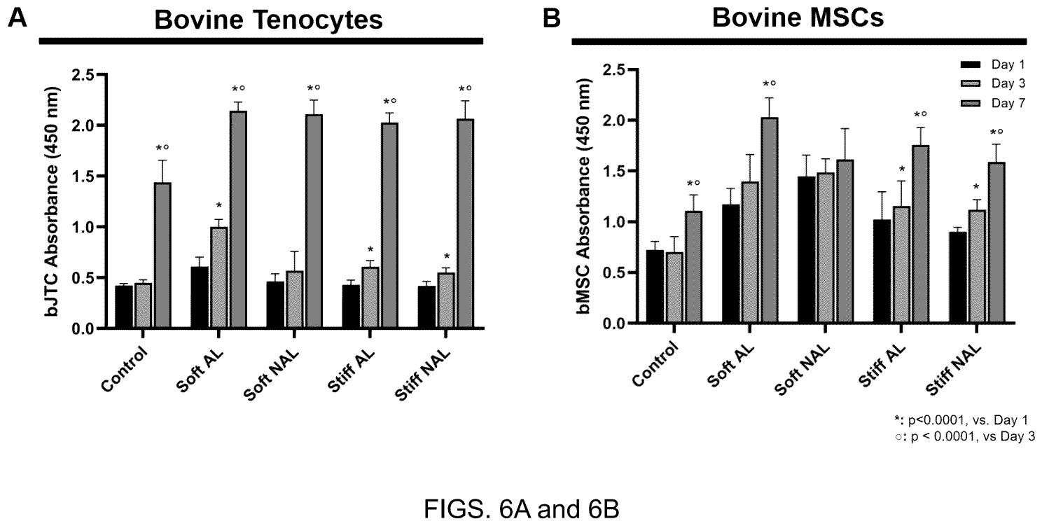

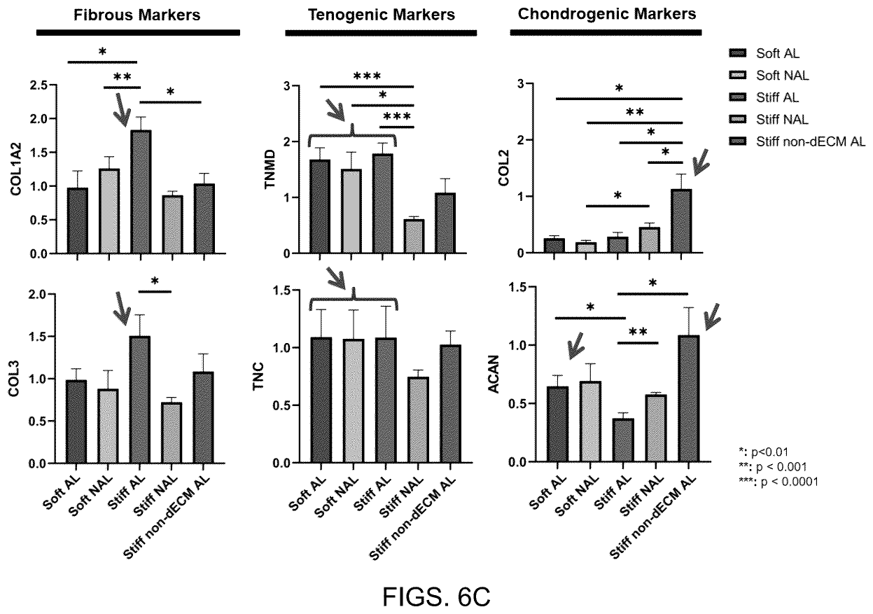

After 3 and 7 days of cell culture, F-actin staining revealed that bovine tenocytes or bovine MSCs attached and spread better on dECM/35% MeHA (soft) or dECM/100% MeHA (stiff) nanofibrous scaffolds than on 100% MeHA alone (FIG. 5A). These results indicated that adding dECM to MeHA improved bovine tendon cell attachment to fibrous scaffolds. FIG. 5B showed that regardless of scaffold stiffness, bovine tenocytes and MSCs had more rounded non-aligned nanofibers with higher aspect ratios. At day 7, a short-term CCK8 assay revealed that dECM groups had higher cell proliferation rates than MeHA alone (non-dECM) groups (FIGS. 6A and 6B). Bovine tendon cells on aligned or stiff scaffolds expressed fibrous and tenogenic markers, whereas nonaligned or soft scaffolds increased the expression of chondrogenic markers (FIG. 6C).

### Example 2—Tunable Decellularized Extracellular Matrix-Based Hydrogel Systems for Meniscus Repair and Regeneration

Introduction

The meniscus plays a pivotal role in knee joint biomechanics, facilitating load bearing and ensuring the even distribution of forces. Regrettably, meniscus injuries are prevalent and severely impact patients' quality of life. In the United States alone, knee arthroscopic surgeries are performed on over 800,000 patients for meniscectomy and 100,000 patients for meniscal repairs annually. However, these surgical interventions often fall short of fully restoring the complex and heterogeneous structure of the meniscus. The meniscus comprises distinct inner and outer zones, each characterized by unique cell phenotypes and extracellular matrix (ECM) compositions. Inadequate repair efforts can result in a substantial increase in the forces transmitted to the articular cartilage, potentially leading to the development of osteoarthritis.

The meniscus exhibits zonal variations in its mechanical properties, including the inner avascular (white-white) zone, intermediate transitional (red-white) zone, and outer vascularized (red-red) zone. These gradient mechanical properties must be faithfully recapitulated in any therapeutic approach aimed at meniscus repair and regeneration. While numerous strategies are under development to address meniscus zone-specific injuries, meniscectomy is becoming a common procedure for precise removal of the damaged meniscal region. However, a majority of patients frequently experience clinical symptoms and functional limitations during daily activities. Intriguingly, these symptoms and limitations often persist even one-year post-surgery. Injured meniscus unfortunately has limited vascularities and hence deficient healing capacity. To overcome the limitations, various biomaterials have been introduced for the meniscal repair and replacement. For instance, meniscus dECM has been introduced as a promising bioactive material for meniscus regeneration given its bioactive properties. Indeed, extracellular matrix (ECM) components change with the tissue development. However, it is still unclear how changes in the ECM components during the development affect meniscus cell phenotype and its regenerative capacity. Thus, age-dependent DEM (extracted from fetal meniscus dECM (FDEM) or adult meniscus dECM (ADEM))-based hydrogels were fabricated, and further evaluated how the age-dependent DEM regulates cellular morphology, proliferation, and gene expression in juvenile bovine meniscal fibrochondrocytes (jbMFC).

Methods

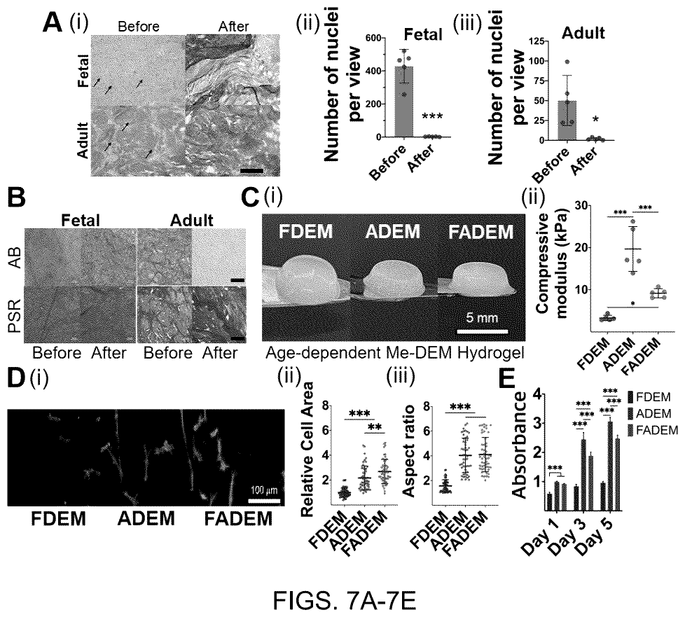

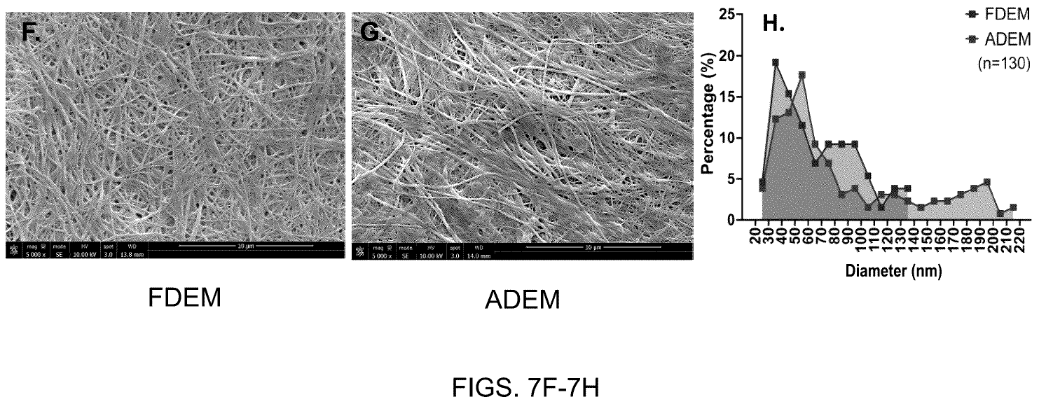

Fetal (3rd trimester) and adult (˜30 months) bovine menisci were collected and decellularized in SDS/Triton-X100 based detergents, and FDEM and ADEM hydrogels were cross-linked under 37° C. for 30 minutes (FIG. 7C(i)). H&E staining was carried out to evaluate the decellularization efficiency and ECM preservation. Alcian Blue (AB) and Picrocirius red (PSR) staining were performed to evaluate the glycosaminoglycan (GAG) and collagen preservation respectively before/after the decellularization. Compressive stiffness of the DEM hydrogel was determined. jbMFC were seeded and cultured on the FDEM, ADEM, or FADEM (50% FDEM/50% ADEM) gelsin basal growth media. Cells stained by Phalloidin were imaged at day 3, and the cell morphology was evaluated using ImageJ. Cell proliferation was assessed by CCK-8. RT-PCR was used to determine the gene expression of Collagen type I or II (Col-I, Col-II), CTGF, Aggrecan (ACAN), SOX9, and MMP in cells at day 5. Proteomic profiles of the FDEM and ADEM were analyzed by mass spectrometry (MS, n=4/group from 4 different donors).

Results

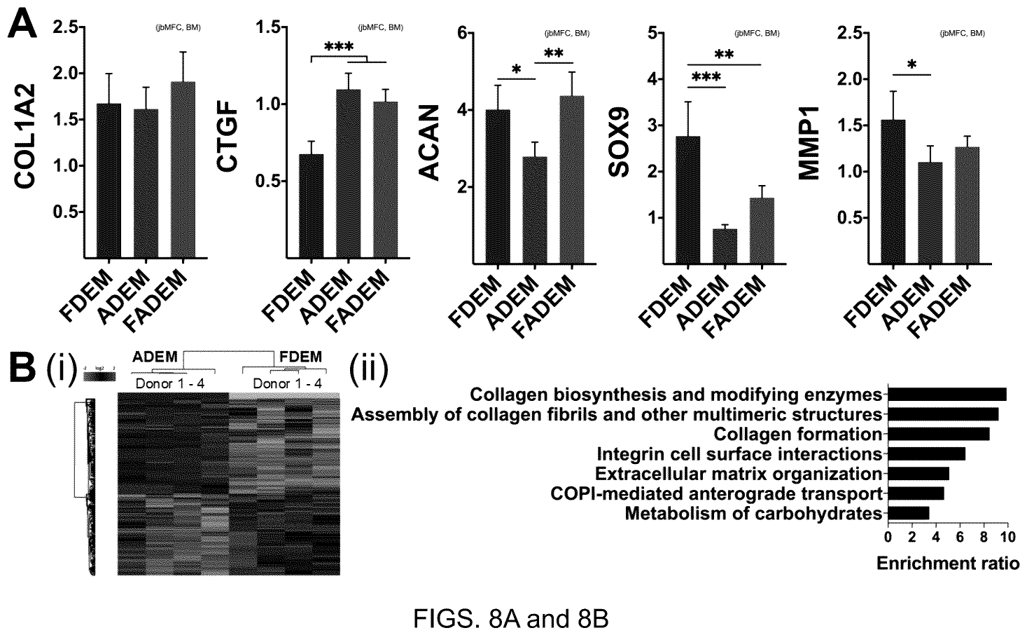

The loss of nuclei in the FDEM or ADEM tissues after decellularization was confirmed (FIG. 7A). While no change in GAG contents in the FDEM after the decellularization was observed, the process decreased the GAG contents in the ADEM, and PSR confirmed that collagen was preserved in both groups (FIG. 7B). The ADEM and FADEM compressive moduli were approximately 6.0 and 2.8 times higher, respectively, than the FDEM (FIG. 7C(ii)). The ADEM and FADEM groups showed increases in cell areas and aspect ratio compared to the FDEM group (FIG. 7D). The cell proliferation rate on the ADEM surface was significantly higher compared with other groups (FIG. 7E). The ADEM enhanced Col-I and CTGF expressions in MFCs (FIG. 8A), however, interestingly cells on the FDEM gels enhanced chondrogenic markers in MFCs (e.g., ACAN and SOX9) and a marker for ECM remodeling (e.g., MMP1) (FIG. 8A), indicating that the age dependent ECM significantly alters jbMFC phenotypes. The MS-based proteomic analysis reveals significant differences in ECM protein components between ADEM and FDEM gels (FIG. 8B(i)). Especially, the analysis shows the ADEM contains more proteins related to collagen synthesis, integrin formation, and cellular metabolism than the FDEM (FIG. 8B(ii)), and it can cause the morphological and genetic alterations of cells on the different hydrogel systems.

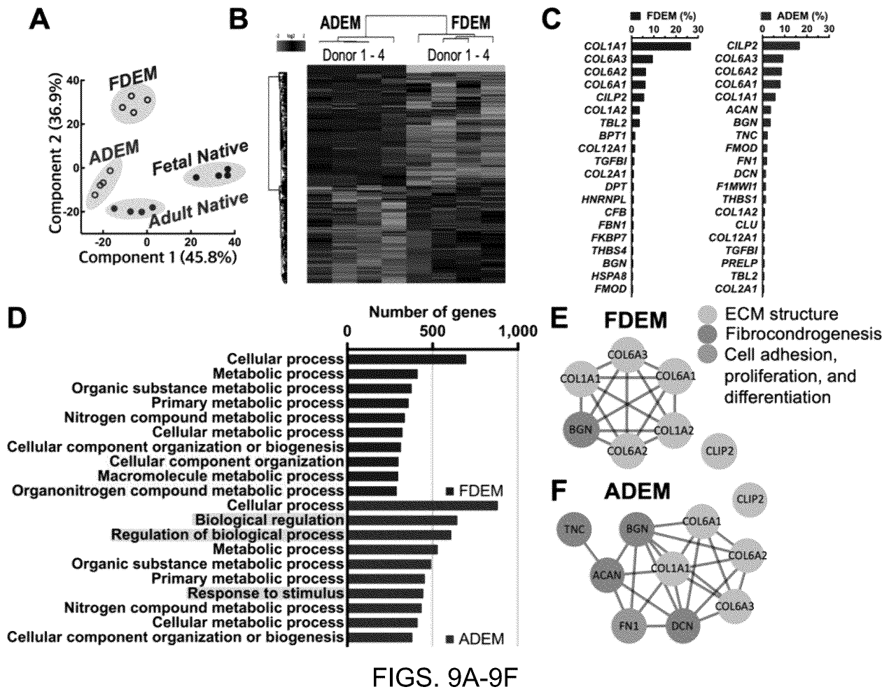

The MS-based proteomic analysis reveals differences in ECM protein components between ADEM and FDEM gels (FIGS. 9A and 9B). In both FDEM and ADEM groups, Collagen-1 (COL1), Collagen-6 (COL6), and Cartilage intermediate layer protein-2 (CILP2) were abundant. Interestingly, Aggrecan (ACAN), Biglycan (BGN), Tenascin-C(TNC), Fibronectin-1 (FN1), and Decorin (DCN) were found only in ADEM groups (FIG. 9C). The proteomic analysis revealed an abundance of proteins related to biological regulation, response to stimuli, and biological processes in the ADEM system as compared to FDEM (FIG. 9D). Among the top 30 proteins constituting the majority of DEM systems, fibrochondrogenesis-related genes were prominent in ADEM (FIG. 9F) compared with those in FDEM (FIG. 9E), including key regulators of cell adhesion, proliferation, and differentiation, including Fibronectin (FN1), forming a highly interconnected network (FIG. 9F).

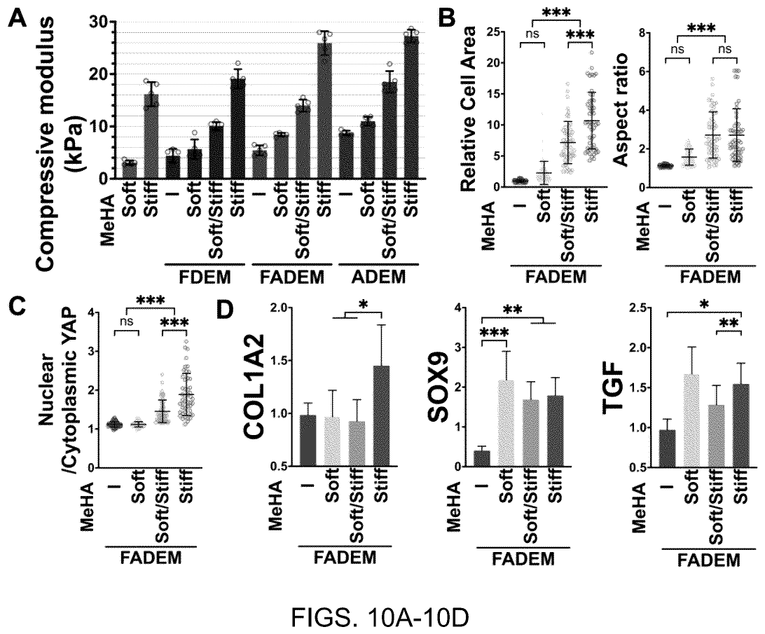

To enhance material injectability and better match specific tissue zones, stiffness-tunable DEM-based MeHA hydrogels were developed, exhibiting a wide range of stiffnesses (FIG. 10A). bMSCs cultured on FADEM-based stiffness-modulated MeHA demonstrated elongation and alignment on the stiff system (FIG. 10B), with elevated YAP nuclear localization (FIG. 10C). Notably, Stiff MeHA-based FADEM groups exhibited heightened COL1A2 expression (FIG. 10D). Interestingly, chondrogenic markers SOX9 and TGF were higher across all MeHA-supplemented groups. The introduction of MeHA to DEM not only boosted chondrogenic gene expression but notably, stiff MeHA stimulated fibrochondrogenic gene expression in MSCs (FIG. 10D).

The innovative stiffness-tunable hydrogel system of the present disclosure, based on the DEM and MeHA, provides an avenue for addressing the clinical challenge of meniscus zone-specific injuries. When injected into a commercially available meniscus tear, this hydrogel system combines various bioactive components, such as bioactive native protein components (e.g., collagen, proteoglycan). Consequently, the system forms a novel material ideally suited for enhancing meniscus cell proliferation and differentiation, fostering meniscus tissue repair, and promoting soft tissue regeneration. The disclosed hydrogel system distinguishes itself from current tissue-engineered biomaterials by granting the ability to precisely adjust hydrogel parameters. This includes fine-tuning biochemical cues and biomechanical stiffness to create an optimal microenvironment for tissue formation. By offering this level of customization, the approach described herein advances meniscus repair and regeneration strategies within the realm of musculoskeletal biology and bioengineering.

In conclusion, novel sophisticated, age-dependent, cross-linkable, tunable based hydrogel systems are provided. This system exhibits a unique capacity for ultraviolet (UV) light activation and tunable stiffness. These characteristics are integral to the creation of a biomimetic, decellularized extracellular matrix (dECM)-based injectable platform tailored to promote the formation of zone-specific meniscus tissue. This work has yielded a versatile and precise platform for tissue engineering applications, particularly in the context of meniscus regeneration. The age-dependent features of the disclosed hydrogel system, coupled with its tunable stiffness and UV light responsiveness, make the system a promising tool for creating biomimetic environments conducive to the development of distinct meniscus tissue zones.

Aspects

The following Aspects are illustrative only and do not limit the scope of the present disclosure or the appended claims. Any part or parts of any one or more Aspects can be combined with any part or parts of any one or more other Aspects.

Aspect 1. A fibrous scaffold that includes (i) one or more modified glycosaminoglycans; and (ii) one or more extracellular matrix (ECM) proteins.

Aspect 2. The fibrous scaffold of Aspect 1, wherein the fibrous scaffold comprises a hydrogel. A hyaluronic acid hydrogel is considered particularly suitable.

Aspect 3. The fibrous scaffold of Aspect 1, wherein the fibrous scaffold comprises at least a first region having a first elastic modulus and at least a second region having a second elastic modulus, the first elastic modulus differing from the second elastic modulus.

Aspect 4. The fibrous scaffold of Aspect 3, wherein the first elastic modulus is about 20 MPa and the second elastic modulus is about 50 MPa.

Aspect 5. The fibrous scaffold of Aspect 1, wherein the fibrous scaffold comprises at least a first region having a first degree of crosslinking and at least a second region having a second degree of crosslinking, the degree of crosslinking differing from the second degree of crosslinking. In this way, a scaffold according to the present disclosure can include a region having a first elastic modulus and a region having a second elastic modulus.

Aspect 6. The fibrous scaffold of Aspect 1, wherein the one or more ECM proteins comprise decellularized ECM obtained from any one or more of tendon tissue, ligament tissue, or cartilaginous tissue.

Aspect 7. The fibrous scaffold of Aspect 1, wherein the one or more ECM proteins comprise any one or more of collagen, elastin, fibronectin, laminin, gelatin, or matricellular protein.

Aspect 8. The fibrous scaffold of Aspect 1, wherein the one or more modified glycosaminoglycans comprise any one or more of hyaluronic acid, chondroitin sulfate, heparin, heparan sulfate, keratan sulfate, or dermatan sulfate.

Aspect 9. The fibrous scaffold of Aspect 1, wherein the one or more modified glycosaminoglycans is modified by methacrylation.

Aspect 10. The fibrous scaffold of Aspect 1, wherein the one or more modified glycosaminoglycans comprises methacrylated hyaluronic acid (MeHA).

Aspect 11. The fibrous scaffold of Aspect 10, wherein the methacrylated hyaluronic acid is methacrylated (i) in a continuous range of from about 35% to about 100% or (ii) in a first region of the fibrous scaffold to about 35% and in a second region of the fibrous scaffold to about 100%, wherein the first region and second region are adjacent and the degree of methacrylation is discrete.

Aspect 12. A method of preparing a fibrous scaffold, comprising:
a) combining a decellularized ECM (dECM) solution and one or more modified glycosaminoglycans to give rise to a polymer solution; and b) generating a fibrous hydrogel scaffold from the polymer solution.

Aspect 13. The method of Aspect 12, wherein the generating comprises i) using any one or more of electrospinning, bioprinting, and injecting the polymer solution and ii) performing one or more crosslinking steps.

Aspect 14. The method of Aspect 12, wherein the fibrous scaffold comprises fibers having a cross-sectional dimension of from about 1 nm to about 1 mm.

Aspect 15. The method of Aspect 12, wherein the dECM comprises any one or more of decellularized collagen elastin, fibronectin, laminin, gelatin, or matricellular protein.

Aspect 16. The method of Aspect 12, wherein a modified glycosaminoglycan comprises methacrylated hyaluronic acid (MeHA).

Aspect 17. The method of Aspect 12, wherein the fibrous hydrogel scaffold comprises at least a first portion and at least a second portion, the first portion having a first elastic modulus and the second portion having a second elastic modulus.

Aspect 18. A method for preparing a tissue biomimetic, comprising: a) seeding a plurality of cells into a fibrous scaffold according to Aspect 1; and b) incubating the seeded fibrous scaffold for a period of time.

Aspect 19. The method of Aspect 18, wherein the plurality of cells comprises any one or more of tenocytes, osteoblasts, chondrocytes, or mesenchymal stem cells.

Aspect 20. An implantable article, comprising the fibrous scaffold of Aspect 1.

The present disclosure also provides methods, the methods comprising implanting a fibrous scaffold according to any one of Aspects 1-11 into a subject. Such implanting can be at, for example, a wound site or a repair site.

What is claimed:

1. A fibrous scaffold, comprising:
(i) one or more modified glycosaminoglycans; and (ii) one or more extracellular matrix (ECM) proteins.

2. The fibrous scaffold of claim 1, wherein the fibrous scaffold comprises a hydrogel.

3. The fibrous scaffold of claim 1, wherein the fibrous scaffold comprises at least a first region having a first elastic modulus and at least a second region having a second elastic modulus, the first elastic modulus differing from the second elastic modulus.

4. The fibrous scaffold of claim 3, wherein the first elastic modulus is about 20 MPa and the second elastic modulus is about 50 MPa.

5. The fibrous scaffold of claim 1, wherein the fibrous scaffold comprises at least a first region having a first degree of crosslinking and at least a second region having a second degree of crosslinking, the degree of crosslinking differing from the second degree of crosslinking.

6. The fibrous scaffold of claim 1, wherein the one or more ECM proteins comprise decellularized ECM obtained from any one or more of tendon tissue, ligament tissue, or cartilaginous tissue.

7. The fibrous scaffold of claim 1, wherein the one or more ECM proteins comprise any one or more of collagen, elastin, fibronectin, laminin, gelatin, or matricellular protein.

8. The fibrous scaffold of claim 1, wherein the one or more modified glycosaminoglycans comprise any one or more of hyaluronic acid, chondroitin sulfate, heparin, heparan sulfate, keratan sulfate, or dermatan sulfate.

9. The fibrous scaffold of claim 1, wherein the one or more modified glycosaminoglycans is modified by methacrylation.

10. The fibrous scaffold of claim 1, wherein the one or more modified glycosaminoglycans comprises methacrylated hyaluronic acid (MeHA).

11. The fibrous scaffold of claim 10, wherein the methacrylated hyaluronic acid is methacrylated (i) in a continuous range of from about 35% to about 100% or (ii) in a first region of the fibrous scaffold to about 35% and in a second region of the fibrous scaffold to about 100%, wherein the first region and second region are adjacent and the degree of methacrylation is discrete.

12. A method of preparing a fibrous scaffold, comprising:
a) combining a decellularized ECM (dECM) solution and one or more modified glycosaminoglycans to give rise to a polymer solution; and
b) generating a fibrous hydrogel scaffold from the polymer solution.

13. The method of claim 12, wherein the generating comprises i) using any one or more of electrospinning, bioprinting, and injecting the polymer solution and ii) performing one or more crosslinking steps.

14. The method of claim 12, wherein the fibrous scaffold comprises fibers having a cross-sectional dimension of from about 1 nm to about 1 mm.

15. The method of claim 12, wherein the dECM comprises any one or more of decellularized collagen elastin, fibronectin, laminin, gelatin, or matricellular protein.

16. The method of claim 12, wherein a modified glycosaminoglycan comprises methacrylated hyaluronic acid (MeHA).

17. The method of claim 12, wherein the fibrous hydrogel scaffold comprises at least a first portion and at least a second portion, the first portion having a first elastic modulus and the second portion having a second elastic modulus.

18. A method for preparing a tissue biomimetic, comprising:
a) seeding a plurality of cells into a fibrous scaffold according to claim 1; and
b) incubating the seeded fibrous scaffold for a period of time.

19. The method of claim 18, wherein the plurality of cells comprises any one or more of tenocytes, osteoblasts, chondrocytes, or mesenchymal stem cells.

20. An implantable article, comprising the fibrous scaffold of claim 1.
